(19) United States
(12) Patent Application Publication
(10) Pub. No.: US 2023/0331858 A1
(43) Pub. Date: Oct. 19, 2023

(54) COMBINATION THERAPY USING AN IL-2 RECEPTOR AGONIST AND AN IMMUNE CHECKPOINT INHIBITOR

(71) Applicant: NEOLEUKIN THERAPEUTICS, INC., Seattle, WA (US)

(72) Inventors: Carl WALKEY, Seattle, WA (US); Jonathan DRACHMAN, Seattle, WA (US); Umut ULGE, Seattle, WA (US); Daniel Adriano SILVA MANZANO, Seattle, WA (US)

(21) Appl. No.: 17/788,598

(22) PCT Filed: Nov. 9, 2020
(86) PCT No.: PCT/US2020/059674

§ 371 Date: Jun. 23, 2022

Related U.S. Application Data
(60) Provisional application No. 63/042,361, filed on Jun. 22, 2020.
(60) Provisional application No. 62/953,362, filed on Dec. 24, 2019.

(51) Int. Cl. C07K 16/28; A61P 35/00

(52) U.S. Cl. CPC C07K 16/2866; A61P 35/00; C07K 16/2818; C07K 16/2827; A61K 2039/505

(57) ABSTRACT

The present disclosure is directed to, inter alia, methods for modulating an immune response in a subject in need thereof using an IL-2 receptor agonist in combination with an immune checkpoint inhibitor.

### CROSS-REFERENCES TO RELATED APPLICATIONS

This application claims priority to U.S. Provisional Pat. Application Serial Nos. 62/953,362 filed Dec. 24, 2019 and 63/042,361 filed Jun. 22, 2020, each incorporated by reference herein in its entirety.

### REFERENCE TO A “SEQUENCE LISTING,” A TABLE, OR A COMPUTER PROGRAM LISTING APPENDIX SUBMITTED ON A COMPACT DISK

This application contains a Sequence Listing which has been submitted electronically in ASCII format and is hereby incorporated by reference in its entirety. Said ASCII copy, created on Nov. 6, 2020, is named 057318_504001WO_Sequence_Listing_ST25.txt and is 28 kilobytes in size.

### BACKGROUND OF THE INVENTION

Monotherapy with checkpoint inhibitors has shown remarkable clinical efficacy in some patients but less clinical efficacy in others. There is a need to develop additional treatments to enhance the effectiveness of checkpoint inhibitors. The present invention meets this and other needs.

### SUMMARY

The present disclosure is directed to, inter alia, methods for modulating an immune response in a subject in need thereof using an IL-2 receptor agonist in combination with an immune checkpoint inhibitor.

### BRIEF DESCRIPTION OF THE DRAWINGS

FIG. 1 displays the anti-tumor activity of a IL-2 receptor agonist (PEGylated IL-2 mimetic) in combination with a PD-1 inhibitor in a CT26 colon cancer model.

FIG. 2 displays the anti-tumor activity of an IL-2 receptor agonist (PEGylated IL-2 mimetic) in combination with a PD-L1 inhibitor in a CT26 colon cancer model.

FIG. 3 displays the anti-tumor activity of an IL-2 receptor agonist (PEGylated IL-2 mimetic) in combination with a PD-1 inhibitor in a MC38 colon cancer model.

FIG. 4 displays the anti-tumor activity of an IL-2 receptor agonist (PEGylated IL-2 mimetic) in combination with a PD-L1 inhibitor in a MC38 colon cancer model.

FIG. 5 displays the anti-tumor activity of an IL-2 receptor agonist (PEGylated IL-2 mimetic) in combination with a PD-1 inhibitor and a CTLA-4 inhibitor (CPI) in a B16F10 mouse melanoma model.

FIG. 6 displays the effect of an IL-2 receptor agonist (PEGylated IL-2 mimetic) on PD-1 expression by CD8+ T cells.

### DETAILED DESCRIPTION OF THE INVENTION

### I. Detailed Description of the Embodiments

The present disclosure is directed to, inter alia, methods for (i) modulating an immune response, (ii) treating cancer, or (iii) inhibiting the proliferation of a tumor in a subject in need thereof using an IL-2 receptor agonist in combination with an immune checkpoint inhibitor.

### A. IL-2 Receptor Agonists

The term IL-2 receptor agonist as used herein refers to a polypeptide capable of activating IL-2 receptor-mediated signaling. In exemplary embodiments, the IL-2 receptor agonist is a long-acting IL-2 receptor agonist. By long-acting, it is meant that the IL-2 receptor agonist has a plasma or serum half-life of 3 hours or greater, preferably 4 hours or greater. In some aspects, the IL-2 receptor agonists will have a serum or plasma half-life of 10 hours or greater or 12 hours or greater. The half-life of a polypeptide refers to the time necessary for the concentration of the polypeptide to decrease by 50% as measured by an appropriate assay. The reduction can be caused by in vivo degradation, clearance, or sequestration of the polypeptide. The half-life of a polypeptide can be determined by any manner known in the art in view of the present disclosure, such as by measuring the concentration of the polypeptide in the blood. For example, to measure the half-life of a polypeptide in vivo, a suitable dose of the polypeptide is administered to a warm-blooded animal (i.e. to a human or to another suitable mammal, such as a mouse, rabbit, rat, pig, dog, or a primate); blood samples or other samples from the animal are collected; the level or concentration of the polypeptide in the sample is determined; and the time until the level or concentration of the polypeptide has been reduced by 50% is calculated based on measured data. See, e.g., Kenneth, A et al., Chemical Half-life of Pharmaceuticals: A Handbook for Pharmacists and Peters et al., Pharmacokinetic analysis: A Practical Approach (1996). As used herein, “an increase in half-life” or “longer half-life” refers to an increase in any one or more of the parameters used to describe the half-life, such as the t1/2-alpha, t1/2-beta and the area under the curve (AUC), as compared to a control. The long-acting nature of the IL-2 receptor agonist can be due to a moiety that it is conjugated or fused to the IL-2 polypeptide. As used herein, the terms “polypeptide”, “protein” or “peptide” refer to any chain of amino acid residues, regardless of its length or post-translational modification (e.g., glycosylation or phosphorylation).

Exemplary IL-2 receptor agonists of the present invention are IL-2 mimetics. IL-2 mimetics are described in Silva et al., Nature 2019 Jan;565(7738):186-191 and U.S. Pat. No. 10,703,791. Exemplary IL-2 mimetics to be used in the present methods induce heterodimerization of IL-2RβƔc, leading to phosphorylation of STAT5. IL-2 mimetics of the present invention bind to the IL-2 receptor βƔc heterodimer (IL-2RβƔc) and typically comprise four helical peptides optionally separated by amino acid linkers.

Neo-2/15 (Silva et al., Nature 2019 Jan;565(7738):186-191 and U.S. Pat. No. 10,703,79) comprises 4 helical domains X1, X2, X3, and X4. Helical domain X1 comprises the amino acid sequence set forth in SEQ ID NO:27 (PKKKIQLHAEHALYDALMILNI); helical domain X2 comprises the amino acid sequence set forth in SEQ ID NO:28 (KDEAEKAKRMKEWMKRIKT); helical domain X3 comprises the amino acid sequence set forth in SEQ ID NO:29 (LEDYAFNFELILEEIARLFESG); and helical domain X4 comprises the amino acid sequence set forth in SEQ ID NO:30 (EDEQEEMANAIITILQSWIFS). In Neo-2/15, these helical domains are in the order X1-X3-X2-X4 and are connected together by amino acid linkers. As a result of Neo-2/15 being a de novo synthesized protein, variants of Neo-2/15 for use as IL-2 receptor agonists in the present invention can have a great deal of variability in the helical domains and amino acid linkers while still retaining the ability to bind to the IL-2 receptor βƔc heterodimer. Methods for determining binding to the IL-2 receptor βƔc heterodimer (IL-2RβƔc) are known in the art as are methods for determining IL-2 receptor agonist activity, e.g., via a STAT5 phosphorylation assay. See, for example, Silva et al., Nature 2019 Jan;565(7738):186-191.

IL-2 receptor agonists to be used in the present methods include IL-2 mimetics comprising an amino acid sequence at least 80%, 85%, 90%, 91%, 92%, 93%, 94%, 95%, 96%, 97%, 98%, 99%, or 100% identical to the amino acid sequence set forth in SEQ ID NO:1 (i.e., the Neo-2/15 polypeptide). In other aspects, IL-2 receptor agonists to be used in the present methods include IL-2 mimetics comprising an amino acid sequence at least 80%, 85%, 90%, 91%, 92%, 93%, 94%, 95%, 96%, 97%, 98%, 99%, or 100% identical to the amino acid sequence as set forth in SEQ ID NO:2. SEQ ID NO:2 is the identical sequence to SEQ ID NO:1 except that the linker amino acids are optional and each amino acid residue of the linker, when present, may comprise any natural or unnatural amino acid. In SEQ ID NO:2, underlined residues (labeled with an X) are linkers and each residue of the linker, when present, may be any amino acid (preferably, a natural amino acid). In exemplary embodiments, the amino acids are natural amino acids. The amino acid linkers, when present, connect the domains. The amino acid linkers may be of any length as deemed appropriate for an intended use. IL-2 mimetics that comprise an amino acid sequence having identity to the amino acid sequence of SEQ ID NO:1 or SEQ ID NO:2 (e.g., 80-99% identity to SEQ ID NO:1 or SEQ ID NO:2) are also referred to herein as Neo-2/15 variants. The term natural amino acid refers to the 20 amino acids that occur naturally in protein. As used herein, the term “unnatural amino acid” refers to an amino acid other than the 20 amino acids that occur naturally in protein. Unnatural amino acids are known in the art.

| | |
|---|---|
| SEQ ID NO:1 NEO 2-15 | PKKKIQLHAEHALYDALMILNIVKTNSPPAEEKLEDYAFNFELILEEIARLFESGDQKDEAEKAKRMKEWMKRIKTTASEDEQEEMANAIITILQSWIFS |
| SEQ ID NO:2 | PKKKIQLHAEHALYDALMILNIXXXXXXXXXXXLEDYAFNFELILEEIARLFESGXXKDEAEKAKRMKEWMKRIKTXXXEDEQEEMANAIITILQSWIFS |

The term “identity”, as used herein in reference to polypeptide sequences, refers to the amino acid sequence identity between two molecules. When an amino acid position in both molecules is occupied by the same amino acid, then the molecules are identical at that position. The identity between two polypeptides is a direct function of the number of identical positions. In general, the sequences are aligned so that the highest order match is obtained (including gaps if necessary). Identity can be calculated using published techniques and widely available computer programs, such as the GCG program package (Devereux et al., Nucleic Acids Res. 12:387, 1984), BLASTP, FASTA (Atschul et al., J. Molecular Biol. 215:403, 1990), etc. Sequence identity can be measured, for example, using sequence analysis software such as the Sequence Analysis Software Package of the Genetics Computer Group at the University of Wisconsin Biotechnology Center (1710 University Avenue, Madison, WI 53705), with the default parameters thereof. If amino acids are added or deleted, it should be done in such a way that doesn’t substantially interfere with presentation of the protein to its binding partner and with secondary structure. Generally, but not necessarily, it is preferable for amino acid substitutions relative to the reference peptide domains to be conservative amino acid substitutions.

As used herein, “conservative amino acid substitution” means a given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising amino acid substitutions can be tested using methods known in the art to confirm that a desired activity, e.g. receptor-binding activity, is retained. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. In some aspects, an amino acid that is not necessary for binding or activity is replaced by cysteine to allow for attachment of a stability moiety.

As used herein, the natural amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

IL-2 receptor agonists of the present invention include variants of Neo-2/15 that have one or more amino acid substitutions in the X1, X2, X3, X4 and/or amino acid linker domains provided that the variants retain Neo-2/15 activity (e.g., the ability to bind to the IL-2 receptor βƔc heterodimer (IL-2RβƔc) leading to phosphorylation of STAT5).

Included in the present invention are Neo-2/15 variants comprising the X1 domain of Neo-2/15 provided that:
the amino acid at position 1 is P or if substituted is A, F, I, L, M, Q, R, S, or W;
the amino acid at position 2 is K or if substituted is A, D, E, G, or V;
the amino acid at position 3 is K or if substituted is D, E, F, or W;
the amino acid at position 4 is K or if substituted is D, E, N, P, R, or W;
the amino acid at position 5 is I or if substituted is D, E, H, K, L, M, or S;
the amino acid at position 6 is Q or if substituted is A, D, E, G, L,P, S, or W;
the amino acid at position 7 is L or if substituted is D, E, Q, Y, or I;
the amino acid at position 8 is H or if substituted is A, F, W,Y, M, or T;
the amino acid at position 9 is A or if substituted is C, F, or P;
the amino acid at position 10 is E or if substituted is C, D, F, K, or P;
the amino acid at position 11 is H or if substituted is D, F, or E;
the amino acid at position 12 is A or if substituted is D, E, P, S, T, or V;
the amino acid at position 13 is L or if substituted is H, I, M, P, R, V, or W;
the amino acid at position 14 is Y or if substituted is F, R, W, or K;
the amino acid at position 15 is D or if substituted is E, N, or Y;
the amino acid at position 16 is A or if substituted is C, L, M, or S;
the amino acid at position 17 is L or if substituted is F, I, M, P, or R;
the amino acid at position 18 is M or if substituted is G, Q, Y, or S;
the amino acid at position 19 is I or if substituted is L, M, P, Q, or V;
the amino acid at position 20 is L or if substituted is A, K, M, Q, R, or S;
the amino acid at position 21 is N or if substituted is G, K, P, R, S, or W;
the amino acid at position 22 is I or if substituted is D, E, K, M, N, W, or Y; and the amino acids positions are in reference to SEQ ID NO:27.

In some such embodiments, 1, 2, 3, 4, or 5 of the following are not true: position 7 is I, position 8 is M or T, position 11 is E, position 14 is K, and position 18 is S. The amino acid positions are in reference to SEQ ID NO:27.

Included in the present invention Neo-2/15 variants comprising the X2 domain of Neo-2/15 provided that:
the amino acid at position 1 is K or if substituted is A, H, L, M, R, S, or V;
the amino acid at position 2 is D or if substituted is A, E, Q, R, S, T, V, W, or Y;
the amino acid at position 3 is E or if substituted is C, G, K, L, N, Q, R, or W;
the amino acid at position 4 is A or if substituted is F, G, N, S, T, V, or Y;
the amino acid at position 5 is E or if substituted is A, G, I, M, R, V, or C;
the amino acid at position 6 is K or if substituted is C, E, L, N, R, or V;
the amino acid at position 7 is A or if substituted is C, E, I, L, S, T, V, or W;
the amino acid at position 8 is K or if substituted is H, L, M, S, T, W, or Y;
the amino acid at position 9 is R or if substituted is A, I, L,M, Q, or S;
the amino acid at position 10 is M or if substituted is A, I, S, W, or Y;
the amino acid at position 11 is K or if substituted is C, I, L, S, or V;
the amino acid at position 12 is E or if substituted is C, K, L, P, Q, R, or T;
the amino acid at position 13 is W or if substituted is A, D, H, or N;
the amino acid at position 14 is M or if substituted is A, C, G, I, L, S, T, or V;
the amino acid at position 15 is K or if substituted is A, E, G, I, L,M, R, or V;
the amino acid at position 16 is R or if substituted is G, H, L, S, T, V, or C;
the amino acid at position 17 is I or if substituted is A, L, or V;
the amino acid at position 18 is K or if substituted is A, C, D, E, G, H, I, M, or S; and
the amino acid at position is 19 is T or if substituted is D, E, G, L, N, or V; and the amino acid positions are in reference to SEQ ID NO:28.

Included in the present invention are Neo-2/15 variants comprising the X3 domain of Neo-2/15 provided that;
the amino acid at position 1 is L or if substituted is A;
the amino acid at position 2 is E or if substituted is D, G, K, M, or T;
the amino acid at position 3 is D or if substituted is E, N, Y, or R;
the amino acid at position 4 is Y or if substituted is C, D, G, T, or F;
the amino acid at position 5 is A or if substituted is F, H, S, V, W, or Y;
the amino acid at position 6 is F or if substituted is A, I, M, T, V, Y, or K;
the amino acid at position 7 is N or if substituted is D, K, S, T, or R;
the amino acid at position 8 is F or if substituted is A, C, G, L, M, S, or V;
the amino acid at position 9 is E or if substituted is C, H, K, L, R, S, T, or V;
the amino acid at position 10 is L or if substituted is F, I, M, Y, or R;
the amino acid at position 11 is I or if substituted is L, N, T, or Y;
the amino acid at position 12 is L or if substituted is F, K, M, S, or V;
the amino acid at position 13 is E or if substituted is A, D, F, G, I, N, P,Q, S, T, or W;
the amino acid at position 14 is E or if substituted is A, F, G, H, S, or V;
the amino acid at position 15 is I or if substituted is C, L, M, V, or W;
the amino acid at position 16 is A or if substituted is D, G, S, T, or V;
the amino acid at position 17 is R or if substituted is H, K, L, or N;
the amino acid at position 18 is L or if substituted is C, D, G, I, Q, R, T, or W;
the amino acid at position 19 is F or if substituted is D, M, N, or W;
the amino acid at position 20 is E or if substituted is A, C, F, G, M, S, or Y;
the amino acid at position 21 is S or if substituted is D, E, G, H, L, M, R, T, V, or W;
the amino acid at position 22 is G or if substituted is A, D, K, N, S, or Y; and the amino acid positions are in reference to SEQ ID NO:29.

In some such embodiments, 1, 2, 3, 4, 5, 6, 7, or all 8 of the following are not true: position 3 is R, position 4 is F, position 6 is K, position 7 is R, position 10 is R, position 11 is N, position 13 is W, and position 14 is G. The amino acid positions are in reference to SEQ ID NO:29

Included in the present invention are Neo-2/15 variants comprising the X4 domain of Neo-2/15 provided that:
the amino acid at position 1 is E or if substituted is D, G, K, or V;
the amino acid at position 2 is D or if substituted is I, M, or S;
the amino acid at position 3 is E or if substituted is G, H, or K;
the amino acid at position 4 is Q or if substituted is E, G, I, K, R, or S;
the amino acid at position 5 is E or if substituted is A, D, G, H, S, or V;
the amino acid at position 6 is E or if substituted is C, D, G, I, M, Q, R,T, or V;
the amino acid at position 7 is M or if substituted is C, E, L, P, R, or T;
the amino acid at position 8 is A or if substituted is F, L, M, or W;
the amino acid at position 9 is N or if substituted is A, G, L, Q, R, or T;
the amino acid at position 10 is A or if substituted is C, D, E, F, H,I, or W;
the amino acid at position 11 is I or if substituted is M, N, S, V, or W;
the amino acid at position 12 is I or if substituted is K, L, S, or V;
the amino acid at position 13 is T or if substituted is C, L, M, R, or S;
the amino acid at position 14 is I or if substituted is L, P, T, or Y;
the amino acid at position 15 is L or if substituted is F, G, I, M, N, or V;
the amino acid at position 16 is Q or if substituted is H, K, or R;
the amino acid at position 17 is S or if substituted is C, F, K, W, or Y;
the amino acid at position 18 is W or if substituted is K, Q, or T;
the amino acid at position 19 is I or if substituted is C, G, or N;
the amino acid at position 20 is F or if substituted is C, G, L, or Y; and
the amino acid at position 21 is S or if substituted is A, F, G, H, or Y; and the amino acid positions are in reference to SEQ ID NO:30.

In some such embodiments, position 19 is not I. In some such aspects, position 19 is C, G, or N. The amino acid positions are in reference to SEQ ID NO:30.

Included in any of these embodiments are Neo-2/15 variants wherein 1, 2, 3, 4, 5, 6, 7, 8, 9, 10, 11, 12, 13, or all 14 of the following are not true: position 7 is I, position 8 is T or M, position 11 is E, position 14 is K, position 18 is S, position 33 is Q, position 36 is R, position 37 is F, position 39 is K, position 40 is R, position 43 is R, position 44 is N, position 46 is W, and position 47 is G wherein the positions are with reference to SEQ ID NO:1. In a further embodiment, one or both of the following are not true: position 68 is I and position 98 is F wherein the positions are with reference to SEQ ID NO:1. When the length of the linkers are of different length, the numbering of residues will change accordingly.

Exemplary IL-2 mimetics for use in the present methods, including Neo-2/15 variants, are conjugated or fused to a stability moiety such as, for example, a water stabilizing moiety such as a PEG-containing moiety. In some aspects, a cysteine residue in the IL-2 mimetic is used for attachment of the PEG moiety. Accordingly, included in the present invention are IL-2 receptor agonists that are IL-2 mimetics comprising an amino acid sequence at least 80%, 85%, 90%, 91%, 92%, 93%, 94%, 95%, 96%, 97%, 98%, 99%, or 100% identical to the amino acid sequence set forth in SEQ ID NO:1or SEQ ID NO:2 except that an amino acid that isn’t necessary for binding is mutated to a cysteine residue for attachment of the stability moiety thereto. In some aspects, the IL-2 mimetic comprises an amino acid sequence as set forth in SEQ ID NO:1 or 2 except that the amino acid at one or more of positions 50, 53, 62, 69, 73, 82, 56, 58, 59, 66, 77, or 85 relative to SEQ ID NO:1 is mutated to a cysteine residue for attachment of a moiety (e.g., PEG-containing moiety) thereto. As referenced above, the skilled practitioner will understand that, in embodiments, wherein the amino acid linkers are of different length, the numbering of residues will change accordingly. As referenced above, the skilled practitioner will understand that, in embodiments, wherein the amino acid linkers are of different length, the numbering of residues will change accordingly. For example, reference to “position 50” relative to SEQ ID NO:1 means the position in SEQ ID NO:2 corresponding to position 50 in SEQ ID NO: 1.

Included in the present invention, are IL-2 mimetics comprising an amino acid sequence at least 25%, 27%, 30%, 35%, 40%, 45%, 50%, 55%, 60%, 65%, 70%, 75%, 80%, 85%, 90%, 95%, 98%, or 100% identical to the amino acid sequence set forth in SEQ ID NO:1 or 2, but for one, two, three, four, five, six, seven, eight, nine, ten, eleven, or all twelve of the following mutations are present:
D56C;
K58C;
D59C;
R66C;
T77C;
E85C;
R50C;
E53C;
E62C;
E69C;
R73C; and/or
E82C.

Included in the present invention, are IL-2 mimetics comprising an amino acid sequence at least 80%, 85%, 90%, 91%, 92%, 93%, 94%, 95%, 96%, 97%, 98%, 99% or 100% identical to the amino acid sequence set forth in SEQ ID NO:1 or 2, but for one, two, three, four, five, six, seven, eight, nine, ten, eleven, or all twelve of the following mutations are present:
D56C;
K58C;
D59C;
R66C;
T77C;
E85C;
R50C;
E53C;
E62C;
E69C;
R73C; and/or
E82C.

This skilled artisan will understand that, with respect to SEQ ID NO:2, positions 56, 58, 59, 66, 77, 85, 50, 53, 62, 69, 73 and 82 referred to above mean the positions in SEQ ID NO: 2 that correspond to positions 56, 58, 59, 66, 77, 85, 50, 53, 62, 69, 73, 82, respectively, in SEQ ID NO:1, and not necessarily the actual positions in SEQ ID NO: 2, which may vary due to the length of the linker.

Exemplary IL-2 mimetics for use in the present invention include those comprising amino acid sequences as set forth in SEQ ID NOs:3-26. In some such embodiments, IL-2 mimetics comprise an amino acid sequence at least 80%, 85%, 90%, 91%, 92%, 93%, 94%, 95%, 96%, 97%, 98%, 99%, or 100% identical to an amino acid sequence selected from any one of SEQ ID NOs: 3-26. Underlined residues are linkers and each residue of the linker (“X”), when present, may be any natural or unnatural amino acid (preferably a natural amino acid). For each IL-2 mimetic below, two SEQ ID NOS are provided: a first SEQ ID NO: that lists the sequence as shown below; and a second SEQ ID NO: that includes the linker positions as variable. It is understood that the position equivalent to R50C (or the listed mutation) will be incorporated in the specified location depending on the length of the linker. In illustrative embodiments, the indicated cysteine is present. IL-2 mimetics that comprise an amino acid sequence having identity to the amino acid sequence of SEQ ID NO:3-26 (e.g., 80-99% identity to SEQ ID NO:3-26) are also referred to herein as Neo-2/15 variants

| | |
|---|---|
| SEQ ID NO:3 NEO 2/15 R50C | PKKKIQLHAEHALYDALMILNIVKTNSPPAEEKLEDYAFNFELILEEIACL FESGDQKDEAEKAKRMKEWMKRIKTTASEDEQEEMANAIITILQSWIFS |
| SEQ ID NO:4 R50C | PKKKIQLHAEHALYDALMILNIXXXXXXXXXXXLEDYAFNFELILEEIACL FESGXXKDEAEKAKRMKEWMKRIKTXXXEDEQEEMANAIITILQSWIFS |
| SEQ ID NO:5 NEO 2-15 E53C | PKKKIQLHAEHALYDALMILNIVKTNSPPAEEKLEDYAFNFELILEEIARL FCSGDQKDEAEKAKRMKEWMKRIKTTASEDEQEEMANAIITILQSWIFS |
| SEQ ID NO:6 E53C | PKKKIQLHAEHALYDALMILNIXXXXXXXXXXXLEDYAFNFELILEEIARL FCSGXXKDEAEKAKRMKEWMKRIKTXXXEDEQEEMANAIITILQSWIFS |
| SEQ ID NO:7 NEO 2-15 D56C | PKKKIQLHAEHALYDALMILNIVKTNSPPAEEKLEDYAFNFELILEEIARL FESGCQKDEAEKAKRMKEWMKRIKTTASEDEQEEMANAIITILQSWIFS |
| SEQ ID NO:8 D56C | PKKKIQLHAEHALYDALMILNIXXXXXXXXXXXLEDYAFNFELILEEIARL FESGCQKDEAEKAKRMKEWMKRIKTXXXEDEQEEMANAIITILQSWIFS |
| SEQ ID NO:9 NEO 2-15 K58C | PKKKIQLHAEHALYDALMILNIVKTNSPPAEEKLEDYAFNFELILEEIARL FESGDQCDEAEKAKRMKEWMKRIKTTASEDEQEEMANAIITILQSWIFS |
| SEQ ID NO:10 K58C | PKKKIQLHAEHALYDALMILNIXXXXXXXXXXXLEDYAFNFELILEEIARL FESGXXCDEAEKAKRMKEWMKRIKTXXXEDEQEEMANAIITILQSWIFS |
| SEQ ID NO:11 NEO 2-15 D59C | PKKKIQLHAEHALYDALMILNIVKTNSPPAEEKLEDYAFNFELILEEIARL FESGDQKCEAEKAKRMKEWMKRIKTTASEDEQEEMANAIITILQSWIFS |
| SEQ ID NO:12 D59C | PKKKIQLHAEHALYDALMILNIXXXXXXXXXXXLEDYAFNFELILEEIARL FESGXXKCEAEKAKRMKEWMKRIKTXXXEDEQEEMANAIITILQSWIFS |
| SEQ ID NO:13 NEO 2-15 E62C | PKKKIQLHAEHALYDALMILNIVKTNSPPAEEKLEDYAFNFELILEEIARL FESGDQKDEACKAKRMKEWMKRIKTTASEDEQEEMANAIITILQSWIFS |
| SEQ ID NO:14 E62C | PKKKIQLHAEHALYDALMILNIXXXXXXXXXXXLEDYAFNFELILEEIARL FESGXXKDEACKAKRMKEWMKRIKTXXXEDEQEEMANAIITILQSWIFS |
| SEQ ID NO:15 NEO 2-15 R66C | PKKKIQLHAEHALYDALMILNIVKTNSPPAEEKLEDYAFNFELILEEIARL FESGDQKDEAEKAKCMKEWMKRIKTTASEDEQEEMANAIITILQSWIFS |
| SEQ ID NO:16 R66C | PKKKIQLHAEHALYDALMILNIXXXXXXXXXXXLEDYAFNFELILEEIARL FESGXXKDEAEKAKCMKEWMKRIKTXXXEDEQEEMANAIITILQSWIFS |

| | |
|---|---|
| SEQ ID NO:17 NEO 2-15 E69C | PKKKIQLHAEHALYDALMILNIVKTNSPPAEEKLEDYAFNFELILEEIARL FESGDQKDEAEKAKRMKCWMKRIKTTASEDEQEEMANAIITILQSWIFS |
| SEQ ID NO:18 E69C | PKKKIQLHAEHALYDALMILNIXXXXXXXXXXXLEDYAFNFELILEEIARL FESGXXKDEAEKAKRMKCWMKRIKTXXXEDEQEEMANAIITILQSWIFS |
| SEQ ID NO:19 NEO 2-15 R73C | PKKKIQLHAEHALYDALMILNIVKTNSPPAEEKLEDYAFNFELILEEIARL FESGDQKDEAEKAKRMKEWMKCIKTTASEDEQEEMANAIITILQSWIFS |
| SEQ ID NO:20 R73C | PKKKIQLHAEHALYDALMILNIXXXXXXXXXXXLEDYAFNFELILEEIARL FESGXXKDEAEKAKRMKEWMKCIKTXXXEDEQEEMANAIITILQSWIFS |
| SEQ ID NO:21 NEO 2-15 T77C | PKKKIQLHAEHALYDALMILNIVKTNSPPAEEKLEDYAFNFELILEEIARL FESGDQKDEAEKAKRMKEWMKRIKTCASEDEQEEMANAIITILQSWIFS |
| SEQ ID NO:22 T77C | PKKKIQLHAEHALYDALMILNIXXXXXXXXXXXLEDYAFNFELILEEIARL FESGXXKDEAEKAKRMKEWMKRIKTCASEDEQEEMANAIITILQSWIFS |
| SEQ ID NO:23 NEO 2-15 E82C | PKKKIQLHAEHALYDALMILNIVKTNSPPAEEKLEDYAFNFELILEEIARL FESGDQKDEAEKAKRMKEWMKRIKTTASEDCQEEMANAIITILQSWIFS |
| SEQ ID NO:24 E82C | PKKKIQLHAEHALYDALMILNIXXXXXXXXXXXLEDYAFNFELILEEIARL FESGXXKDEAEKAKRMKEWMKRIKTXXXEDCQEEMANAIITILQSWIFS |
| SEQ ID NO:25 NEO 2-15 E85C | PKKKIQLHAEHALYDALMILNIVKTNSPPAEEKLEDYAFNFELILEEIARL FESGDQKDEAEKAKRMKEWMKRIKTTASEDEQECMANAIITILQSWIFS |
| SEQ ID NO:26 E85C | PKKKIQLHAEHALYDALMILNIXXXXXXXXXXXLEDYAFNFELILEEIARL FESGXXKDEAEKAKRMKEWMKRIKTXXXEDEQECMANAIITILQSWIFS |

Accordingly, provided herein are IL-2 receptor agonists for use in the present invention that are IL-2 mimetics comprising an amino acid sequence at least 80% identical to an amino acid sequence selected from the group consisting SEQ ID NOs. 3-26. Provided herein are IL-2 receptor agonists for use in the present invention that are IL-2 mimetics comprising an amino acid sequence at least 85% identical to an amino acid sequence selected from the group consisting SEQ ID NOs. 3-26. Provided herein are IL-2 receptor agonists for use in the present invention that are IL-2 mimetics comprising an amino acid sequence at least 90% identical to an amino acid sequence selected from the group consisting SEQ ID NOs. 3-26. Provided herein are IL-2 receptor agonists for use in the present invention that are IL-2 mimetics comprising an amino acid sequence at least 95% identical to an amino acid sequence selected from the group consisting SEQ ID NOs. 3-26. Provided herein are IL-2 receptor agonists for use in the present invention that are IL-2 mimetics comprising an amino acid sequence selected from the group consisting SEQ ID NOs. 3-26. In any of these embodiments, the polypeptide may be an IL-2 mimetic as described herein, wherein 1, 2, 3, 4, 5, 6, 7, 8, 9, 10, 11, 12, 13, or all 14 of the following are not true: position 7 is I, position 8 is T or M, position 11 is E, position 14 is K, position 18 is S, position 33 is Q, position 36 is R, position 37 is F, position 39 is K, position 40 is R, position 43 is R, position 44 is N, position 46 is W, and position 47 is G. In a further embodiment, one or both of the following are not true: position 68 is I and position 98 is F. The positions are in reference to SEQ ID NO:1. In illustrative embodiments, the indicated cysteine is present.

Exemplary IL-2 mimetics of the present invention are linked to other compounds to promote an increased half-life in vivo, e.g., PEG compounds. A “PEG” is a poly(ethylene glycol) molecule which is a water-soluble polymer of ethylene glycol. PEGs can be obtained in different sizes and can also be obtained commercially in chemically activated forms that are derivatized with chemically reactive groups to enable covalent conjugation to proteins. Linear PEGs are produced in various molecular weights, such as PEG polymers of weight-average molecular weights of 5,000 daltons, 10,000 daltons, 20,000 daltons, 30,000 daltons, and 40,000 daltons. Branched PEG polymers have also been developed. Methods for conjugating polypeptides to PEG are known in the art and can be used herein. Commonly-used activated PEG polymers are those derivatized with maleimide or iodoacetamide groups (for coupling to thiols such as cysteine residues). For example, in some embodiments, addition of polyethylene glycol (“PEG”) containing moieties may comprise attachment of a PEG group linked to a maleimide group (e.g., “PEG-MAL”) to a cysteine residue of the polypeptide. Suitable examples of a PEG group linked to maleimide group (“PEG-MAL”) include, but are not limited to, methoxy PEG-MAL 5 kD; methoxy PEG-MAL 20 kD; methoxy (PEG)2-MAL 40 kD; methoxy PEG(MAL)2 5 kD; methoxy PEG(MAL)2 20 kD; methoxy PEG(MAL)2 40 kD; or any combination thereof. See also U.S. Pat. No. 8,148,109. The skilled artisan will be able to use the methods described herein or alternative methods to design long-acting IL-2 receptor agonists for use in the present invention, including by attaching a PEG group to an IL-2 mimetic of the present invention, whether via maleimide or another attachment strategy.

In some embodiments, the IL-2 receptor agonist to be used in the present methods comprises the amino acid sequence set forth in SEQ ID NO:13 (NEO 2-15 E62C), wherein the cysteine at position 62 is PEGylated. The polyethylene group can be attached via any suitable attachment chemistry, including, for example, with maleimide (e.g., maleimide-modified PEG, PEG-MAL 5 kD; PEG-MAL 20 kD; or PEG-MAL 40 kD). In some embodiments, the PEGylation is with PEG-MAL 30 kD. In some embodiments, the PEGylation is with PEG-MAL 40 kD. In some embodiments, the range for repeating PEG units in the PEGylated peptide is about 800-1000. In some embodiments, the average number of repeating PEG units in the PEGylated peptide is about 850-950. One of skill in the art will understand that PEG portions can be linear or branched.

In some embodiments, the IL-2 receptor agonist to be used in the present methods comprises the amino acid sequence set forth in SEQ ID NO:23 (NEO 2-15 E82C), wherein the cysteine at position 82 is PEGylated. The polyethylene group can be attached via any suitable attachment chemistry, including, for example, with maleimide (e.g., maleimide-modified PEG, PEG-MAL 5 kD; PEG-MAL 20 kD; or PEG-MAL 40 kD). In some embodiments, the PEGylation is with PEG-MAL 30 kD. In some embodiments, the PEGylation is with PEG-MAL 40 kD. In some embodiments, the range for repeating PEG units in the PEGylated peptide is about 800-1000. In some embodiments, the average number of repeating PEG units in the PEGylated peptide is about 850-950. One of skill in the art will understand that PEG portions can be linear or branched.

In some embodiments, the IL-2 receptor agonist to be used in the present methods comprises the amino acid sequence set forth in SEQ ID NO:17 (NEO 2-15 E69C), wherein the cysteine at position 69 is PEGylated. The polyethylene group can be attached via any suitable attachment chemistry, including, for example, with maleimide (e.g., maleimide-modified PEG, PEG-MAL 5 kD; PEG-MAL 20 kD; or PEG-MAL 40 kD). In some embodiments, the PEGylation is with PEG-MAL 30 kD. In some embodiments, the PEGylation is with PEG-MAL 40 kD. In some embodiments, the range for repeating PEG units in the PEGylated peptide is about 800-1000. In some embodiments, the average number of repeating PEG units in the PEGylated peptide is about 850-950. One of skill in the art will understand that PEG portions can be linear or branched.

In some embodiments, the IL-2 receptor agonist to be used in the present methods comprises the amino acid sequence set forth in SEQ ID NO:19 (NEO 2-15 R73C), wherein the cysteine at position 73 is PEGylated. The polyethylene group can be attached via any suitable attachment chemistry, including, for example, with maleimide (e.g., maleimide-modified PEG, PEG-MAL 5 kD; PEG-MAL 20 kD; or PEG-MAL 40 kD). In some embodiments, the PEGylation is with PEG-MAL 30 kD. In some embodiments, the PEGylation is with PEG-MAL 30 kD. In some embodiments, the PEGylation is with PEG-MAL 40 kD. In some embodiments, the range for repeating PEG units in the PEGylated peptide is about 800-1000. In some embodiments, the average number of repeating PEG units in the PEGylated peptide is about 850-950. One of skill in the art will understand that PEG portions can be linear or branched.

The polypeptides and peptide domains disclosed herein may include additional residues at the N-terminus, C-terminus, or both; these additional residues are not included in determining the percent identity of the polypeptides or peptide domains of the disclosure relative to the reference polypeptide. Such residues may be any residues suitable for an intended use, including but not limited to detection tags (i.e.: fluorescent proteins, antibody epitope tags, etc.), adaptors, ligands suitable for purposes of purification (His tags, etc.), other peptide domains that add functionality to the polypeptides, etc. Residues suitable for attachment of such groups may include, for example, cysteine, lysine or p-acetylphenylalanine residues or can be tags, such as amino acid tags suitable for reaction with transglutaminases as disclosed in U.S. Pat. Nos. 9,676,871 and 9,777,070.

### B. Immune Checkpoint Inhibitors

Immune checkpoints are signaling proteins that stimulate or inhibit an immune response. Compositions that target immune checkpoints modulate these proteins to alter an individual’s natural immune response. The immune checkpoint inhibitors described herein are those that inhibit or block immune checkpoint molecules that help keep immune responses in check (e.g., they keep cells, such as T cells, from killing cancer cells). When these immune checkpoint molecules are blocked or inhibited, the “brakes” on the immune system are released and cells, such as T cells, are better able to locate and kill cancer cells. Accordingly, an immune checkpoint inhibitor, as used herein, is a molecule that inhibits the ability of an immune checkpoint molecule to suppress the immune system. In some aspects, the inhibitor can directly bind the immune checkpoint molecule, a molecule controlling the expression of the immune checkpoint molecule, or a ligand of the immune checkpoint molecule that mediates the activity of the immune checkpoint molecule. The inhibitor or antagonist may be an antibody (including a humanized or human antibody), a small molecule, a peptide, or a nucleic acid (e.g., an antisense molecule, or a single- or double-stranded RNAi molecule).

In some aspects, the checkpoint inhibitor a biologic therapeutic or a small molecule. In some embodiments, the checkpoint inhibitor is a monoclonal antibody (e.g., a chimeric antibody, a humanized antibody or fully human antibody) or a fusion protein. In some embodiments, the checkpoint inhibitor inhibits a ligand of a checkpoint inhibitor selected from the group consisting of CLTA-4, PD-1, or PD-L1. In some embodiments, the checkpoint inhibitor is a PD-L1, PD-1, or CTLA-4 inhibitor.

In some embodiments, the immune checkpoint inhibitor is a CTLA-4 antagonist, such as an antagonistic CTLA-4 antibody. In some embodiments, the CLTA-4 antagonist is selected from YERVOY (ipilimumab), tremelimumab, AGEN1884, and AGEN2041.

In some embodiments, the immune checkpoint inhibitor is a PD-1 antagonist, such as an antagonistic PD-1 antibody. Suitable PD-1 antibodies include, for example, OPDIVO (nivolumab), KEYTRUDA (pembrolizumab), or MEDI-0680 (AMP-514). The immuno-oncology agent may also include pidilizumab (CT-011), though its specificity for PD-1 binding has been questioned. Another approach to target the PD-1 receptor is the recombinant protein composed of the extracellular domain of PD-L2 (B7-DC) fused to the Fc portion of IgG1, called AMP-224.

In some embodiments, the immune checkpoint inhibitor is a PD-L1 antagonist, such as an antagonistic PD-L1 antibody. Suitable PD-L1 antibodies include, for example, atezolizumab, avelumab, durvalumab, BMS-936559, MPDL3280A (RG7446; W02010/077634), and MSB0010718C.

In some embodiments, two immune checkpoint inhibitors are used in combination with the IL-2 receptor agonist, e.g., a PD-L1 antagonist or PD-1 antagonist in combination with a CTLA-4 antagonist.

The antibody immune checkpoint inhibitors of the present disclosure can be prepared using known methods in the art. For example, human monoclonal antibodies of this disclosure can be prepared using SCID mice into which human immune cells have been reconstituted such that a human antibody response can be generated upon immunization. Such mice are described in, for example, U.S. Pat. Nos. 5,476,996 and 5,698,767 to Wilson et al. The immune checkpoint inhibitors of the present disclosure can also be formulated to retard the degradation of the agent or to minimize the immunogenicity of the antibody. A variety of techniques are known in the art to achieve this purpose.

### C. Methods of Treatment

The present disclosure provides, inter alia, methods for modulating an immune response in a subject by administering to the subject an IL-2 receptor agonist of the present disclosure in combination with an immune checkpoint inhibitor. As used herein, the term “subject” refers to an animal, preferably a mammal, more preferably a human.

As used herein, an “immune response” being modulated refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments of the compositions and methods described herein, an immune response being modulated is T-cell mediated. Methods of measuring an immune response are known in the art and include, for example, measuring pro-inflammatory cytokines such as IL-6, IL-12 and TNF-alpha as well as co-stimulatory molecules, such as CD80, CD86, and chemokine receptor.

In a further aspect, the present disclosure provides methods for treating cancer, comprising administering to a subject in need thereof the combination therapy regimen as described herein. As used herein, “treat” or “treating” means accomplishing one or more of the following: (a) reducing the size or volume of tumors and/or metastases in the subject; (b) limiting any increase in the size or volume of tumors and/or metastases in the subject; (c) increasing survival; (d) reducing the severity of symptoms associated with cancer; (e) limiting or preventing development of symptoms associated with cancer; and (f) inhibiting worsening of symptoms associated with cancer.

The methods can be used to treat cancer, including but not limited to, colon cancer, melanoma, renal cell cancer, head and neck squamous cell cancer, gastric cancer, urothelial carcinoma, Hodgkin lymphoma, non-small cell lung cancer, small cell lung cancer, hepatocellular carcinoma, pancreatic cancer, Merkel cell carcinoma colorectal cancer, acute myeloid leukemia, acute lymphoblastic leukemia, chronic lymphocytic leukemia, non-Hodgkin lymphoma, multiple myeloma, ovarian cancer, cervical cancer, breast cancer, liver cancer, renal cell carcinoma, melanoma, and any tumor types selected by a diagnostic test, such as microsatellite instability, tumor mutational burden, PD-L1 expression level, or the immunoscore assay (as developed by the Society for Immunotherapy of Cancer). In some aspects, the cancer is a solid tumor or liquid tumor. In some aspects, the cancer is one that is resistant to monotherapy with checkpoint inhibitors. In some aspects, the cancer is one that is sensitive to monotherapy with checkpoint inhibitors.

In a further aspect, the present disclosure provides methods for inhibiting the proliferation of a tumor in subject, comprising administering to the subject in need thereof the combination therapy regimen as described herein. The tumors can be associated with a solid cancer or a liquid cancer. In some aspects, the tumor is associated with colon cancer, melanoma, renal cell cancer, head and neck squamous cell cancer, gastric cancer, urothelial carcinoma, Hodgkin lymphoma, non-small cell lung cancer, small cell lung cancer, hepatocellular carcinoma, pancreatic cancer, Merkel cell carcinoma colorectal cancer, acute myeloid leukemia, acute lymphoblastic leukemia, chronic lymphocytic leukemia, non-Hodgkin lymphoma, multiple myeloma, ovarian cancer, cervical cancer, breast cancer, liver cancer, renal cell carcinoma, or melanoma.

In some embodiments, the methods described herein include one or more additional agents in addition to the IL-2 receptor agonist and the immune checkpoint inhibitors described above. For example, in certain cancers such as melanoma, a further agent such as an anti-TYRP1 antibody can be used. In some embodiments, the anti-TYPR1 antibody is TA99.

The term “therapeutically effective amount” means the amount of the subject peptide, antibody, or other active agent that will elicit the biological or medical response of a cell, tissue, system, or animal, such as a human, that is being sought by the researcher, veterinarian, medical doctor or other treatment provider.

The term “inhibit” or “inhibition of” means to reduce by a measurable amount, or to prevent entirely. The term inhibition as used herein can refer to an inhibition or reduction of at least 10%, at least 15%, at least 20%, at least 25%, at least 30%, at least 40%, at least 50%, at least 60%, at least 70%, or at least 80%.

As used herein the term synergy or synergistic effect when used in connection with a description of the efficacy of a combination of agents, means any measured effect of the combination which is greater than the effect predicted from a sum of the effects of the individual agents (i.e., greater than an additive effect). In some embodiments, the rate of tumor growth or tumor size (e.g., the rate of change of the size (e.g., volume, mass) of the tumor) is used to determine whether a combination of drugs is synergistic (e.g., the combination of drugs is synergistic when the rate of tumor growth is slower than would be expected if the combination of drugs produced an additive effect). In some embodiments, survival time is used to determine whether a combination of drugs is synergistic (e.g., a combination of drugs is synergistic when the survival time of a subject or population of subjects is longer than would be expected if the combination of drugs produced an additive effect).

### D. Methods of Administration of Combination Therapy

The methods described herein include administering to a subject (e.g., a human subject) a therapeutically effective amount of an IL-2 receptor agonist and a therapeutically effective amount of one or more immune checkpoint inhibitors. In some embodiments, the one or more immune checkpoint inhibitors is a PD-1 inhibitor. In some embodiments, the one or more immune checkpoint inhibitors is a PD-L1 inhibitor. In some embodiments, the one or more immune checkpoint inhibitors is a CTLA-4 inhibitor. In some embodiments, the one or more immune checkpoint inhibitors are a PD-1 inhibitor and a CTLA-4 inhibitor. In some aspects, the combination of therapeutic agents acts synergistically to affect the treatment or prevention of cancer or the modulation of an immune response or the inhibition of the proliferation of tumor cells.

Depending on the disease status and the subject’s condition, the peptides, antibodies, and formulations of the present disclosure may be administered by any suitable means. Typically peptide and antibody therapies are administered parenterally (e.g., intramuscular, intraperitoneal, intravenous, ICV, intracisternal injection or infusion, subcutaneous injection, or implant). Other modes of administration may be suitable for the present methods as well, for example, oral administration. In addition, the peptides and antibodies may be formulated, alone or together, in suitable dosage unit formulations containing conventional nontoxic pharmaceutically acceptable carriers, adjuvants and vehicles appropriate for each rouse of administration.

It will be understood, that the specific dose level and frequency of dosage for any particular patient may be varied and will depend upon a variety of factors including the activity of the active agents employed, the metabolic stability and length of action of these agents, the age, body weight, hereditary characteristics, general health, sex, diet, mode and time of administration, rate of excretion, drug combination, the severity of the particular condition, and the host undergoing therapy. A suitable dosage range for the IL-2 receptor agonist may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. In other embodiments, the recommended dose could be based on weight/m² (i.e. body surface area), and/or it could be administered at a fixed dose (e.g., .05-100 mg). In some aspects, a suitable dose range for the IL-2 mimetic is from 0.5 ug/kg to 30 ug/kg or from 1 ug/kg to 10 ug/kg or 8 ug/mg.

Generally, the optimal amount the IL-2 receptor agonist and the checkpoint inhibitor that is effective in the methods provided herein (e.g., treatment of cancer) can be determined by standard clinical techniques. In addition, in vitro assays may optionally be employed to help identify optimal dosage ranges. The precise dose to be employed in the formulation will also depend on the route of administration, and the stage of malignancy, and should be decided according to the judgment of the practitioner and each patient’s circumstances. Effective doses may be extrapolated from dose-response curves derived from in vitro or animal model test systems.

In some embodiments, the IL-2 receptor agonist and the checkpoint inhibitor will be administered to a subject at the Maximal Tolerable Dose (MTD) or the Optimal Biological Dose (OBD). It is within the art to determine MTD or OBD. In some aspects, the IL-2 receptor agonist will be provided at its MTD or OBD and the checkpoint inhibitor will be dosed at 50%-100%, preferably at 50% to 90% of the MTD or OBD. Alternatively, the checkpoint inhibitor will be dosed at its MTD or OBD and the IL-2 receptor agonist will be dosed at at 50%-100%, preferably at 50% to 90% of the MTD or OBD. In some aspects, both the IL-2 receptor agonist and the checkpoint inhibitor will be dosed at 60% to 90% of the MTD or OBD

As used in this invention, the combination regimen can be given simultaneously or can be given in a staggered regimen, with the checkpoint inhibitor being given at a different time during the course of therapy than IL-2 receptor agonist. This time differential may range from several minutes, hours, days, weeks, or longer between administration of the two agents. Therefore, the term combination does not necessarily mean administered at the same time or as a unitary dose, but that each of the components are administered during a desired treatment period to provide the desired effect. The agents may also be administered by different routes.

### E. Kits

In some aspects, provided herein are kits containing an IL-2 receptor agonist and one or more immune check-point inhibitors. A kit can contain a pharmaceutical composition containing an IL-2 receptor agonist and one or more pharmaceutical composition containing immune checkpoint inhibitors, e.g., an anti-PD-1 inhibitor, ante-PD-L1 and/or an anti-CTLA-4 inhibitor. In some instances, the kit includes written materials e.g., instructions for use of the peptide, antibody or pharmaceutical compositions thereof. Without limitation, the kit may include buffers, diluents, filters, needles, syringes, and package inserts with instructions for performing any methods disclosed herein.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

### II. EXAMPLES

### Example 1 Preparation of Exemplary PEGylated IL-2 Mimetic

Neo-2/15 stocks with a single E62C mutation (SEQ ID NO:19) were dialyzed into phosphate buffer, pH7.0 and adjusted to 1.0-2.0 mg/ml. TCEP was added at a molar ratio of 10:1 to protein and incubated for 10 minutes at RT to reduce disulfides. Maleimide-modified PEG40k (PEG40k-MA) or PEG30k (PEG30k-MA) powder was added directly to the reduced protein solution at a molar ratio of 10:1 PEG:cysteine and incubated for 2 hours with stirring. Aliquots for SDS-PAGE were taken directly from the reaction mixture. These data demonstrate the rapid, spontaneous, and near-quantitative formation of covalent linkages between PEG40k-MA or PEG30k-MA and Neo-2/15 cysteine mutants in the expected stoichiometry. This PEGylated IL-2 mimetic is used in the examples below.

### Example 2: Combination Study of PEGylated IL-2 Mimetic With Checkpoint Inhibition in a CT26 Mouse Colon Cancer Model

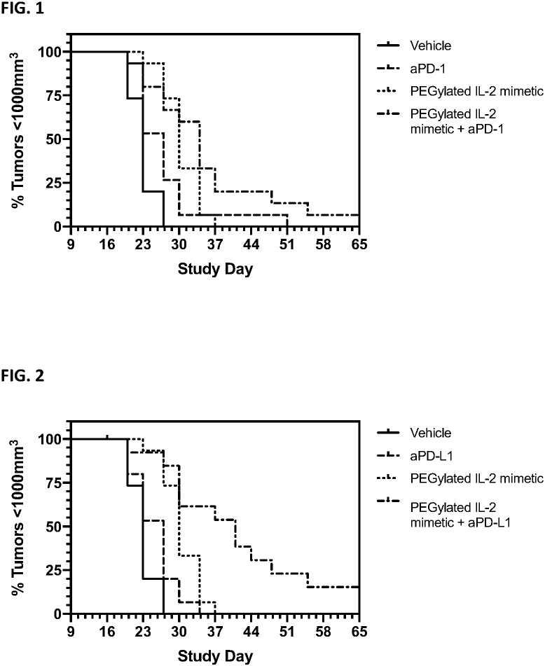

This study was carried out to evaluate the combined anti-tumor activity of an IL-2R agonist with antibody inhibitors of the PD-1 (aPD-1; clone RMP1-14 ) and PD-L1 (aPD-L1; clone 10F.9G2) immune checkpoints in the CT26 mouse colon cancer model. CT26 tumor cells were maintained in culture media. The cells were harvested in the exponential growth phase. Each BALB/c mouse was inoculated subcutaneously in the right rear flank with CT26 tumor cells (5e5) in 0.1 mL PBS. Randomization into treatment groups was performed when the mean tumor size reached 80-120 mm^3. Treatment was initiated immediately post grouping. Vehicle is a non-specific antibody that was dosed ip at 10 mg/kg biweekly for 6 doses. The IL-2R agonist (PEGylated IL-2 mimetic) was dosed iv at 60 ug/kg QW for 2 doses. Anti-PD-1 or anti-PD-L1 were dosed ip at 10 mg/kg biweekly for 6 doses. Tumor volumes were measured twice per week in two dimensions using a caliper. Tumor volume was expressed in mm^3 using the formula V=((LxW)xW)/2. The percentage tumors that are less than 1000 mm³ are shown at days 16, 23, 30, 37, 44, 51, and 58 for (i) a control arm, anti-PD-1 monotherapy arm, PEGylated IL-2 mimetic monotherapy arm, and anti-PD-1/PEGylated IL-2 mimetic combination therapy arm (See FIG. 1 and Table 1) and for (i) a control arm, anti-PD-L1 monotherapy arm, PEGylated IL-2 mimetic monotherapy arm, and anti-PD-L1/PEGylated IL-2 mimetic combination therapy arm (See FIG. 2 and Table 2).

TABLE 1

% Tumors < 1000 mm3

| Day | Vehicle | aPD-1 | PEGylated IL-2 mimetic | PEGylated IL-2 mimetic + aPD-1 |
|---|---|---|---|---|
| 16 | 100 | 100 | 100 | 100 |
| 23 | 20 | 53.3 | 93.3 | 93.3 |
| 30 | 0 | 6.7 | 33.3 | 60 |
| 37 | 0 | 0 | 0 | 20 |
| 44 | 0 | 0 | 0 | 20 |
| 51 | 0 | 0 | 0 | 13.3 |
| 58 | 0 | 0 | 0 | 6.7 |
| Median Survival | 23 | 27 | 30 | 34 |

TABLE 2

% Tumors < 1000 mm3

| Day | Vehicle | aPD-L1 | PEGylated IL-2 mimetic | PEGylated IL-2 mimetic + aPD-L1 |
|---|---|---|---|---|
| 16 | 100 | 100 | 100 | 100 |
| 23 | 20 | 53.3 | 93.3 | 92.3 |
| 30 | 0 | 6.7 | 33.3 | 61.5 |
| 37 | 0 | 0 | 0 | 53.8 |
| 44 | 0 | 0 | 0 | 30.8 |
| 51 | 0 | 0 | 0 | 23.1 |
| 58 | 0 | 0 | 0 | 15.4 |
| Median Survival | 23 | 27 | 30 | 41 |

This Median survival (MS) for mice treated with vehicle was 23 days. MS was increased by 4 days with aPD-L1 treatment, MS was increased by 7 days with low-dose PEGylated IL-2 mimetic treatment, and by 18 days for the combination of PEGylated IL-2 mimetic treatment and aPD-L1. The increase in MS for the combination of PEGylated IL-2 mimetic treatment and aPD-L1 is synergistic when compared to each treatment alone.

With respect to the combination study of PEGylated IL-2 mimetic with aPD-1 treatment, MS showed additive effects, whereas inhibition of tumor growth showed synergy at various time points. At day 30, the aPD-1 arm showed that 6.7% of tumors were less than 1000 mm3, the PEGylated IL-2 mimetic arm showed that 33.3% of tumors were less than 1000 mm3, but the combination arm showed that 61.5% of tumors were less than 1000 mm3.

### Example 3: Combination Study of PEGylated IL-2 Mimetic With Checkpoint Inhibition in a MC38 Mouse Colon Cancer Model

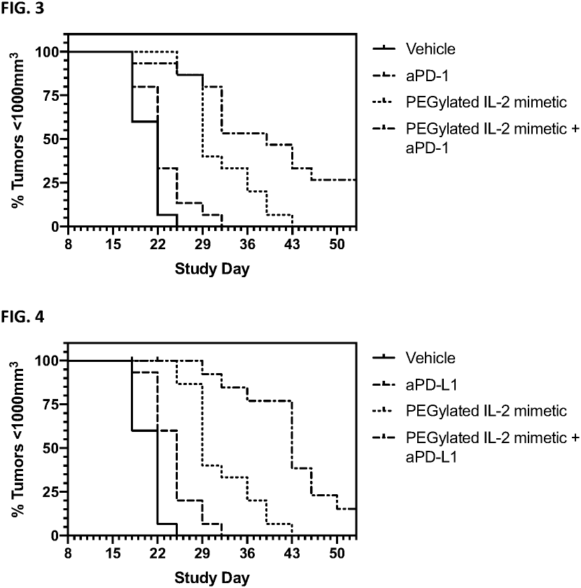

This study was carried out to evaluate the combined anti-tumor activity of an IL-2R agonist with antibody inhibitors of the PD-1 (aPD-1; clone RMP1-14) and PD-L1 (aPD-L1; clone 10F.9G2) immune checkpoints in the MC38 mouse colon cancer model. MC38 tumor cells were maintained in culture media. The cells were harvested in the exponential growth phase. Each C57BL6 mouse was inoculated subcutaneously in the right rear flank with MC38 tumor cells (1e6) in 0.1 mL PBS. Randomization into treatment groups was performed when the mean tumor size reached 80-120 mm^3. Treatment was initiated immediately post grouping. Vehicle is a non-specific antibody that was dosed ip at 10 mg/kg biweekly for 6 doses. IL-2R agonist was dosed iv at 60 ug/kg QW for 2 doses. Anti-PD-1 or anti-PD-L1 were dosed at 10 mg/kg ip biweekly for 6 doses. Tumor volumes were measured twice per week in two dimensions using a caliper. Tumor volume was expressed in mm^3 using the formula V=((LxW)xW)/2. The percentage tumors that are less than 1000 mm³ are shown at days 15, 22, 29, 36, 43, and 50 for (i) a control arm, anti-PD-1 monotherapy arm, PEGylated IL-2 mimetic monotherapy arm, and anti-PD-1/ PEGylated IL-2 mimetic combination therapy arm (See FIG. 3 and Table 3) and for (i) a control arm, anti-PD-L1 monotherapy arm, PEGylated IL-2 mimetic monotherapy arm, and anti-PD-L1/ PEGylated IL-2 mimetic combination therapy arm (See FIG. 4 and Table 4).

TABLE 3

% Tumors < 1000 mm3

| Day | Vehicle | aPD-1 | PEGylated IL-2 mimetic | PEGylated IL-2 mimetic + aPD-1 |
|---|---|---|---|---|
| 15 | 100 | 100 | 100 | 100 |
| 22 | 6.7 | 33.3 | 100 | 93.3 |
| 29 | 0 | 6.7 | 40 | 80 |
| 36 | 0 | 0 | 20 | 53.3 |
| 43 | 0 | 0 | 0 | 33.3 |
| 50 | 0 | 0 | 0 | 26.7 |
| Median Survival | 22 | 22 | 29 | 39 |

TABLE 4

% Tumors < 1000 mm3

| Day | Vehicle | aPD-L1 | PEGylated IL-2 mimetic | PEGylated IL-2 mimetic + aPD-L1 |
|---|---|---|---|---|
| 15 | 100 | 100 | 100 | 100 |
| 22 | 6.7 | 60 | 100 | 100 |
| 29 | 0 | 6.7 | 40 | 92.3 |
| 36 | 0 | 0 | 20 | 69.2 |
| 43 | 0 | 0 | 0 | 38.5 |
| 50 | 0 | 0 | 0 | 15.4 |
| Median Survival | 22 | 25 | 29 | 43 |

This Median survival (MS) for mice treated with vehicle was 22 days. MS was not increased with aPD-1 treatment but was increased by 3 days with aPD-L1 treatment. MS was increased by 7 days with low-dose PEGylated IL-2 mimetic treatment, while MS was increased by 17 and 21 days for the combination of PEGylated IL-2 mimetic treatment + aPD-1 and PEGylated IL-2 mimetic treatment + aPD-L1, an improvement of 10 and 14 days over PEGylated IL-2 mimetic treatment alone. The increase in MS for the combination of PEGylated IL-2 mimetic treatment and aPD-1 or aPD-L1 is synergistic when compared to each treatment alone.

### Example 4: Combination Study of PEGylated IL-2 Mimetic With Checkpoint Inhibition in a B16F10 Mouse Melanoma Model

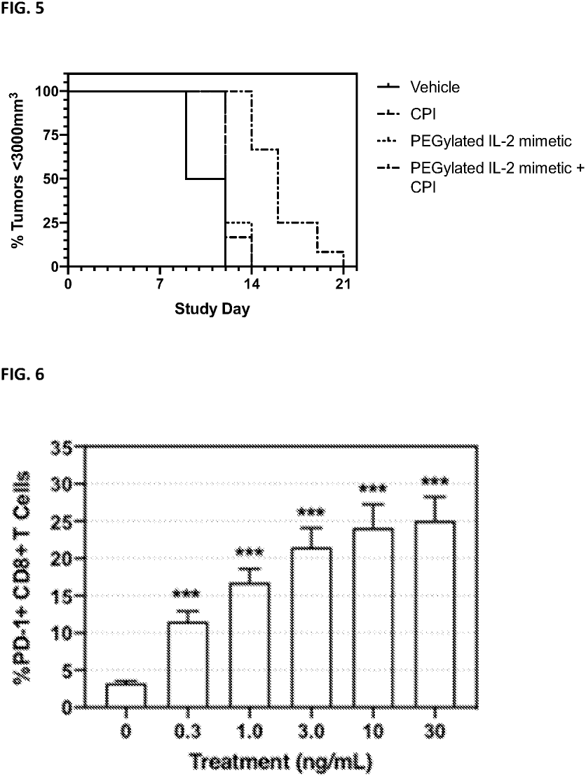

This study was carried out to evaluate the combined anti-tumor activity of a PEGylated IL-2 mimetic with antibody inhibitors of the PD-1 (aPD-1; clone RMP1-14) immune checkpoint in combination with the CTLA-4 inhibitor (aCTLA-4; clone 9D9) in the B16F10 mouse melanoma model. B16F10 tumor cells were maintained in culture media. The cells were harvested in the exponential growth phase. Each C57BL6 mouse was inoculated subcutaneously in the right rear flank with B16F10 tumor cells (2e5) in 0.1 mL PBS. Randomization into treatment groups was performed when the mean tumor size reached ~80 mm^3 with 12 animals per group. Treatment was initiated immediately post grouping. Vehicle is a non-specific antibody that was dosed ip at 10 mg/kg biweekly for 6 doses. PEGylated IL-2 mimetic was dosed iv at 275 ug/kg QW for 2 doses. Anti-PD-1 in combination with the anti-CTLA-4 (noted as CPI in Table 5 below and FIG. 5) was dosed at 10 mg/kg ip biweekly for 6 doses. Tumor volumes were measured three times per week. Tumor volume was expressed in mm^3 using the formula V=((LxW)xW)/2. See FIG. 5

TABLE 5

% Tumors < 3000 mm3

| Day | Vehicle | CPI | PEGylated IL-2 mimetic | PEGylated IL-2 mimetic + CPI |
|---|---|---|---|---|
| 0 | 100 | 100 | 100 | 100 |
| 9 | 50 | 100 | 100 | 100 |
| 12 | 0 | 16.7 | 25 | 100 |
| 14 | 0 | 0 | 0 | 66.7 |
| 16 | 0 | 0 | 0 | 25 |
| 19 | 0 | 0 | 0 | 8.3 |
| 21 | 0 | 0 | 0 | 0 |
| Median Survival | 10.5 | 12 | 12 | 16 |

This Median survival (MS) for mice treated with vehicle was 10.5 days. MS was increased by 1.5 days with anti-PD-1 in combination with the anti-CTLA-4 1 treatment and by 1.5 days with PEGylated 1L-2 mimetic, and by 5.5 days with the combination therapy (anti-PD1, anti-CTLA-4 and PEGylated IL-2 mimetic). The increase in MS for the combination is synergistic when compared to each treatment alone.

### Example 5: The Effect of PEGylated IL-2 Mimetic on PD-1 Expression by CD8+ Cells

To evaluate the effect of PEGylated IL-2 mimetic stimulation on the expression of the immune checkpoint receptor, PD-1, by lymphocytes, PBMCs from 10 human donors were isolated and treated with PEGylated IL-2 mimetic (0-30 ng/ml) before being washed, stained and analyzed by flow cytometry. PEGylated IL-2 mimetic stimulation led to a concentration-dependent increased in PD-1 expression by CD8+ T cells, consistent with induced proliferation, suggesting that combining the PEGylated IL-2 mimetic with a PD-1 inhibitor may overcome immune checkpoint-mediated CD8+ T cell inhibition. See FIG. 6

What is claimed is:

1. A method of (i) modulating an immune response or (ii) treating cancer in a subject, said method comprising administering to the subject an effective amount of an IL-2 receptor agonist and one or more immune checkpoint inhibitors.

2. A method of treating cancer in a subject, said method comprising administering to the subject an effective amount of an IL-2 receptor agonist and one or more immune checkpoint inhibitors.

3. The method of claim 1 or claim 2, wherein the cancer is selected from the group consisting of melanoma and renal cell carcinoma.

4. The method of claim 1 or 2 wherein the cancer is colon cancer.

5. The method of claim 1 or 2 wherein the cancer is colorectal cancer, breast cancer, lung cancer, a sarcoma, head and neck cancer, liver cancer or bladder cancer.

6. The method of claim 1 or 2 wherein the cancer is a solid tumor.

7. A method for inhibiting the proliferation of a tumor in a subject, said method comprising administering to the subject an effective amount of an IL-2 receptor agonist and one or more immune checkpoint inhibitors.

8. The method of claim wherein the tumor is from colorectal cancer, breast cancer, lung cancer, a sarcoma, head and neck cancer, liver cancer bladder cancer, melanoma, or renal cell carcinoma.

9. The method of any one of claims 1 to 8, wherein the IL-2 receptor agonist comprises an amino acid sequence at least 80%, 85%, 90%, 91%, 92%, 93%, 94%, 95%, 96%, 97%, 98%, 99%, or 100% identical to an amino acid sequence selected from the group consisting of SEQ ID NO:3 to SEQ ID NO:26.

10. The method of claim 9, wherein the IL-2 receptor agonist comprises an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:3, 5, 7, 9, 11, 13, 15, 17, 19, 21, 23, and 25.

11. The method of claim 10, wherein the IL-2 receptor agonist comprises an amino acid sequence at least 80%, 85%, 90%, 91%, 92%, 93%, 94%, 95%, 96%, 97%, 98%, 99%, or 100% identical to SEQ ID NO:13, provided that the cysteine at position 62 is present.

12. The method of claim 10 wherein the IL-2 receptor agonist comprises an amino acid sequence at least 80%, 85%, 90%, 91%, 92%, 93%, 94%, 95%, 96%, 97%, 98%, 99%, or 100% identical to SEQ ID NO:23, provided that the cysteine at position 82 is present.

13. The method of claim 10, wherein the IL-2 receptor agonist comprises an amino acid sequence at least 80%, 85%, 90%, 91%, 92%, 93%, 94%, 95%, 96%, 97%, 98%, 99%, or 100% identical to SEQ ID NO:17 provided that the cysteine at position 69 is present.

14. The method of any one of claim 10, wherein the IL-2 receptor agonist comprises an amino acid sequence at least 80%, 85%, 90%, 91%, 92%, 93%, 94%, 95%, 96%, 97%, 98%, 99%, or 100% identical to SEQ ID NO:19, provided that the cysteine at position 73 is present.

15. The method of any one of claims 1 to 14, wherein the IL-2 receptor agonist further comprises a polyethylene glycol (“PEG”) containing moiety.

16. The method of claim 15, wherein the PEG-containing moiety is linked at a cysteine residue in the polypeptide.

17. The method of 15 wherein (i) the IL-2 receptor agonist comprises the amino acid sequence set forth in SEQ ID NO:13 and the cysteine at position 62 is linked to the PEG-containing moiety, (ii) the IL-2 receptor agonist comprises the amino acid sequence set forth in SEQ ID NO:23 and the cysteine at position 82 is linked to PEG-containing moiety, (iii) the IL-2 receptor agonist comprises the amino acid sequence set forth in SEQ ID NO:17 and the cysteine at position 69 is linked to the PEG-containing moiety, or (iv) the IL-2 receptor agonist comprises the amino acid sequence set forth in SEQ ID NO:19 and the cysteine at position 73 is linked to the PEG-containing moiety.

18. The method of claim 16 or 17 wherein polyethylene glycol is linked via a maleimide group to the cysteine residue.

19. The method of any one of claims 15 to 18, wherein the number of repeating PEG units in the PEG-containing moiety is about 800-1000.

20. The method of any one of claims 15 to 18, wherein the number of repeating PEG units in the PEG-containing moiety is about 850-950.

21. The method of any one of claims 1 to 20 wherein the immune checkpoint inhibitor is an antagonist of CTLA-4, PD-1, or PD-L1.

22. The method of any one of claims 1 to 21 wherein the immune checkpoint inhibitor is an antibody.

23. The method of claim 21, wherein the antagonist of CLTA-4 is an antibody selected from the group consisting of Ipilimumab, Tremelimumab, AGEN1884, and AGEN2041.

24. The method of claim 21, wherein the antagonist of PD-1 is an antibody selected from the group consisting of nivolumab, pembrolizumab, and MEDI-0680.

25. The method of claim 21, wherein the antagonist of PD-L1 is an antibody selected from the group consisting of durvalumab, BMS-936559, MPDL3280A, and MSB0010718C.

26. The method of claim 21, wherein the antagonist of PD-L1 is an antibody selected from the group consisting of atezolizumab and avelumab.

27. The method of any one of claims 1 to 26, wherein an antagonist of CTLA-4 and an antagonist of PD-1 or PD-L1 is administered to the subject.

28. The method of any one of claims 1 to 27 wherein the combination of the IL-2 receptor agonist and one or more immune checkpoint inhibitors provides a synergistic effect in the treatment of the cancer or modulation of immune response or the inhibition of the proliferation of tumor cells.


SEQUENCE LISTING (30 sequences)

SEQ ID NO: 1 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTTASEDEQEEMANAIITILQSWIFS

SEQ ID NO: 2 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKRMKEWMKRIKTXXXEDEQEEMANAIITILQSWIFS

SEQ ID NO: 3 - protein, 100 aa, Artificial sequence
  Synthetic construct
PKKKIQLHAEHALYDALMILNIVKTNSPPAEEKLEDYAFNFELILEEIACLFESGDQKDEAEKAKRMKEWMKRIKTTASEDEQEEMANAIITILQSWIFS

SEQ ID NO: 4 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIACLFESGXXKDEAEKAKRMKEWMKRIKTXXXEDEQEEMANAIITILQSWIFS

SEQ ID NO: 5 - protein, 100 aa, Artificial sequence
  Synthetic construct
PKKKIQLHAEHALYDALMILNIVKTNSPPAEEKLEDYAFNFELILEEIARLFCSGDQKDEAEKAKRMKEWMKRIKTTASEDEQEEMANAIITILQSWIFS

SEQ ID NO: 6 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFCSGXXKDEAEKAKRMKEWMKRIKTXXXEDEQEEMANAIITILQSWIFS

SEQ ID NO: 7 - protein, 100 aa, Artificial sequence
  Synthetic construct
PKKKIQLHAEHALYDALMILNIVKTNSPPAEEKLEDYAFNFELILEEIARLFESGCQKDEAEKAKRMKEWMKRIKTTASEDEQEEMANAIITILQSWIFS

SEQ ID NO: 8 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGCQKDEAEKAKRMKEWMKRIKTXXXEDEQEEMANAIITILQSWIFS

SEQ ID NO: 9 - protein, 100 aa, Artificial sequence
  Synthetic construct
PKKKIQLHAEHALYDALMILNIVKTNSPPAEEKLEDYAFNFELILEEIARLFESGDQCDEAEKAKRMKEWMKRIKTTASEDEQEEMANAIITILQSWIFS

SEQ ID NO: 10 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXCDEAEKAKRMKEWMKRIKTXXXEDEQEEMANAIITILQSWIFS

SEQ ID NO: 11 - protein, 100 aa, Artificial sequence
  Synthetic construct
PKKKIQLHAEHALYDALMILNIVKTNSPPAEEKLEDYAFNFELILEEIARLFESGDQKCEAEKAKRMKEWMKRIKTTASEDEQEEMANAIITILQSWIFS

SEQ ID NO: 12 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKCEAEKAKRMKEWMKRIKTXXXEDEQEEMANAIITILQSWIFS

SEQ ID NO: 13 - protein, 100 aa, Artificial sequence
  Synthetic construct
PKKKIQLHAEHALYDALMILNIVKTNSPPAEEKLEDYAFNFELILEEIARLFESGDQKDEACKAKRMKEWMKRIKTTASEDEQEEMANAIITILQSWIFS

SEQ ID NO: 14 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEACKAKRMKEWMKRIKTXXXEDEQEEMANAIITILQSWIFS

SEQ ID NO: 15 - protein, 100 aa, Artificial sequence
  Synthetic construct
PKKKIQLHAEHALYDALMILNIVKTNSPPAEEKLEDYAFNFELILEEIARLFESGDQKDEAEKAKCMKEWMKRIKTTASEDEQEEMANAIITILQSWIFS

SEQ ID NO: 16 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKCMKEWMKRIKTXXXEDEQEEMANAIITILQSWIFS

SEQ ID NO: 17 - protein, 100 aa, Artificial sequence
  Synthetic construct
PKKKIQLHAEHALYDALMILNIVKTNSPPAEEKLEDYAFNFELILEEIARLFESGDQKDEAEKAKRMKCWMKRIKTTASEDEQEEMANAIITILQSWIFS

SEQ ID NO: 18 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKRMKCWMKRIKTXXXEDEQEEMANAIITILQSWIFS

SEQ ID NO: 19 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKCIKTTASEDEQEEMANAIITILQSWIFS

SEQ ID NO: 20 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKRMKEWMKCIKTXXXEDEQEEMANAIITILQSWIFS

SEQ ID NO: 21 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTCASEDEQEEMANAIITILQSWIFS

SEQ ID NO: 22 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKRMKEWMKRIKTCASEDEQEEMANAIITILQSWIFS

SEQ ID NO: 23 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTTASEDCQEEMANAIITILQSWIFS

SEQ ID NO: 24 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKRMKEWMKRIKTXXXEDCQEEMANAIITILQSWIFS

SEQ ID NO: 25 - protein, 100 aa, Artificial sequence
  Synthetic construct
PKKKIQLHAEHALYDALMILNIVKTNSPPAEEKLEDYAFNFELILEEIARLFESGDQKDEAEKAKRMKEWMKRIKTTASEDEQECMANAIITILQSWIFS

SEQ ID NO: 26 - protein, 100 aa, Artificial sequence
  Synthetic construct
  MISC_FEATURE (1)..(100): Xaa can be any amino acid and may be present or absent
PKKKIQLHAEHALYDALMILNIXXXXXXXXXXXLEDYAFNFELILEEIARLFESGXXKDEAEKAKRMKEWMKRIKTXXXEDEQECMANAIITILQSWIFS

SEQ ID NO: 27 - protein, 22 aa, Artificial sequence
  Synthetic construct
LEDYAFNFELILEEIARLFESG

SEQ ID NO: 28 - protein, 22 aa, Artificial sequence
  Synthetic construct
PKKKIQLHAEHALYDALMILNI

SEQ ID NO: 29 - protein, 19 aa, Artificial sequence
  Synthetic construct
KDEAEKAKRMKEWMKRIKT

SEQ ID NO: 30 - protein, 21 aa, Artificial sequence
  Synthetic construct
EDEQEEMANAIITILQSWIFS